(19) United States
(12) Patent Application Publication
(10) Pub. No.: US 2005/0085814 A1
(43) Pub. Date: Apr. 21, 2005

(54) Dynamizable orthopedic implants and their use in treating bone defects

(76) Inventors: Michael C. Sherman, Memphis, TN (US); Jeff R. Justis, Gulf Breeze, FL (US)

Correspondence Address: WOODARD, EMHARDT, MORIARTY, MCNETT & HENRY LLP, BANK ONE CENTER/TOWER, 111 MONUMENT CIRCLE, SUITE 3700, INDIANAPOLIS, IN, 46204-5137, US

(21) Appl. No.: 10/690,451

(22) Filed: Oct. 21, 2003

(51) Int. Cl. A61B 17/58

(52) U.S. Cl. 606/61

(57) ABSTRACT

The present invention generally relates to bone fixation devices and methods for promoting arthrodesis of bone defects. The bone fixation devices can provide sufficient support to the bone defect while allowing bone ingrowth and minimizing the risk to stress shield and/or pseudo-arthrodesis. The bone fixation devices include a degradable component that allows the device to gradually transfer the load from the supporting member to the growing bone structure in vivo.

### BACKGROUND OF THE INVENTION

The present invention relates generally to orthopedic devices for promoting bone fusion and methods for treating orthopedic defects using the orthopedic devices.

The spine is composed of both rigid and flexible elements, forming a complex structure that can readily accommodate a wide range of motions and adjust to a wide range of loads. Unfortunately, the spine, like any complex physiological structure, is also vulnerable to disease, injury, and congenital deficiencies, all of which can cause defects to the spine and, in particular, to the vertebral body and intervertebral discs. Spinal disease, injury, and deformity may have a disastrous impact on patient well being, ranging from acute pain to chronic debilitating pain, and, in the most severe cases, partial or complete paralysis.

Some of the most common pathologies of spinal defects include fractured, diseased, or decayed vertebral bodies, torn or stretched ligaments, and damaged or diseased intervertebral discs.

Common treatments for defective vertebrae include joining or fusing fractured bone segments or portions together to stabilize the affected parts and removing the affected vertebrae, either in part or in whole. Classically, the damaged disc is excised, the adjacent vertebrae are mechanically joined together, and oftentimes bone is grafted into the region particularly in the disc space between the two vertebrae to promote fusion of the adjacent vertebrae. The vertebrae can be mechanically joined using a prosthetic device such as a bone plate that is attached to the adjacent vertebrae with bone screws. The bone plate eliminates disparate motion between the two bone portions to allow arthrodesis.

It is particularly important that the prosthetic device not stress shield the new bone growth and permit a weakened juncture or pseudoarthrodesis between the bone portions or adjacent vertebrae to be fused. It is known that for load bearing bone members, stronger, denser bone tissue results when the new bone growth occurs under pressure. The problem arises when and how to determine the amount of pressure or force desirable to develop a strong junction between the bone portions. The bone portions should be secured and supported during bone growth. However, the optimum support necessary for desired bone growth may vary over time as the bony juncture or bridge develops between the bone portions.

Similarly, torn and/or structural ligaments can be treated by initially securing/immobilizing the ligaments. This can be accomplished using either or both internal and external prosthetic devices to augment or replace the stability lost as a result of the damaged ligaments. Further, the treated ligaments can be susceptible to repeated injury. Consequently, it may be desirable to augment the treated ligament by implanting a prosthesis or device that allows limited movement of the affected ligaments, i.e., stretching and rotation of the ligaments. Current treatment methods do not allow for an implanted device to initially secure/immobilize the ligaments and then allow limited movement of the same without a subsequent surgical revisitation.

In light of the above, there is a continuing need for devices and treatments that stabilize and support damaged bone tissue and bony structures and connecting tissue, provide variable loads to growing bone, as well as a measure of flexible support to injury or disease prone bones and connecting tissue. The present invention addresses this need and provides other benefits and advantages in a novel and nonobvious manner.

### BRIEF SUMMARY OF THE INVENTION

The present invention relates to orthopedic devices, the manufacture and use thereof. Various aspects of the invention are novel, nonobvious, and provide various advantages. While the actual nature of the invention covered herein can only be determined with reference to the claims appended hereto, certain forms and features, which are characteristic of the preferred embodiments disclosed herein, are described briefly as follows.

In one form, the present invention provides an orthopedic device for securing two or more bone portions. The device comprises an elongate member configured for engagement to the two or more bone portions and allowing translational or rotational movement for a first one of the two or more bone portions relative to a second one of the two or more bone portions; a reinforcing component composed of a biodegradable material and engaged to the elongate member to inhibit the translational or rotational movement for a first one of the two or more bone portions relative to a second one of the two or more bone portions; and at least one bone fastener for fixedly securing the elongate member to at least one of the two or more bone portions. The orthopedic device can be used to treat a variety of bone defects including but not limited to: bone fractures, diseased bone tissue, spinal diseases, diseased/damaged vertebrae, torn or stretched ligaments and the like.

In another form, the present invention provides a method for treating a bone defect. The method comprises providing an orthopedic device including an elongate member configured to be deformable in vivo, and a reinforcing component encasing at least a portion of the elongate member. The reinforcing component comprises a biodegradable material, which is formulated to inhibit deformation of the elongate member. The first end of the elongate member can be secured to first bony structure and the second end of the elongate structure can be secured to a second bony structure. The secured device can support and effectively immobilize the two bone portions relative to each other. In vivo, the reinforcing component can be degraded and be eliminated either in whole or in part from the device. This effectively transfers at least a portion of the biomechanical load and support to the treated site, in general, and to new tissue and bone growth, in particular. Further particularly for articulating joints and if desired, the treated site can then be allowed at least a limited amount of movement, i.e. translation and/or rotation. The secured devices without the reinforcing component can be allowed to remain in place indefinitely.

Further objects, features, aspects, forms, advantages and benefits shall become apparent from the description and drawings contained herein.

### BRIEF DESCRIPTION OF THE DRAWINGS

FIG. 1 is perspective view of one embodiment of a bone fixation device in accordance with the present invention.

FIG. 2 is plan view of an elongate member for use in the bone fixation device of FIG. 1.

FIG. 3 is a plan view of an alternate embodiment of bone fixation device in accordance with the present invention.

FIG. 4 is a perspective view of an elongate member for use in the bone fixation device of FIG. 3.

FIG. 5 is a perspective view of yet another embodiment of a bone fixation device having a bendable portion in accordance with the present invention.

FIG. 6 is a perspective view of an elongate member for use in the bone fixation device of FIG. 5.

FIG. 7 is a perspective view of one embodiment of an orthopedic rod including a rigid biodegradable material supporting a portion of the rod in accordance with the present invention.

FIG. 8 is one embodiment of a hollow orthopedic rod with an inner core of reinforcing material in accordance with the present invention.

FIG. 9 is a perspective view of another embodiment of an orthopedic rod with a movable reinforcing element for use in accordance with the present invention.

FIG. 10 is a perspective view of the orthopedic rod of FIG. 9 with the movable reinforcing element positioned to allow the rod to be deformed.

FIG. 11 is a perspective view of one embodiment of the bone fixation device of FIG. 1 secured to adjacent vertebrae.

FIG. 12 is a perspective view of the bone fixation device of FIG. 1 absent the reinforcing component.

### DESCRIPTION OF THE PREFERRED EMBODIMENTS

For the purposes of promoting an understanding of the principles of the invention, reference will now be made to the embodiments illustrated herein and specific language will be used to describe the same. It will nevertheless be understood that no limitation of the scope of the invention is thereby intended. Any alterations and further modifications in the described devices, systems, and treatment methods, and any further applications of the principles of the invention as described herein, are contemplated as would normally occur to one skilled in the art to which the invention relates.

In preferred embodiments, the present invention provides an implantable orthopedic device or prosthesis to facilitate support and repair of defective bone structures and/or connective tissue. The defective bone structures can be the result of damaged, traumatized, and/or diseased tissue. By use of the term orthopedic device, it is intending to include within its meaning a device that can be used defective, diseased and/or damaged tissue of the muscular/skeletal system(s).

The devices of the present invention can provide initial support and/or fixation of selected bone structures. After a selected period of time or under certain conditions, the amount and nature of the support/fixation can vary to facilitate a desirable treatment. For example, the variable or dynamizable support develops new, strong bone tissue minimizing the risk of pseudoarthrodesis.

The devices of the present invention also find advantageous use to treat connecting tissue such as ligaments. The devices can augment the connecting tissue. After a predetermined period of time or condition, the device can allow limited movement, either translational and/or rotational, of the connection tissue and/or attached bone structures as desired. For example, if the natural connecting tissue is elastic (i.e., cartilage or ligaments), the device can serve to limit or restrict the overall length or amount that the connecting tissue stretches. This restriction can vary depending upon the length of time or preselected conditions that the device has been implanted. The following description specifically describes non-limiting, specific embodiments for use with the present invention.

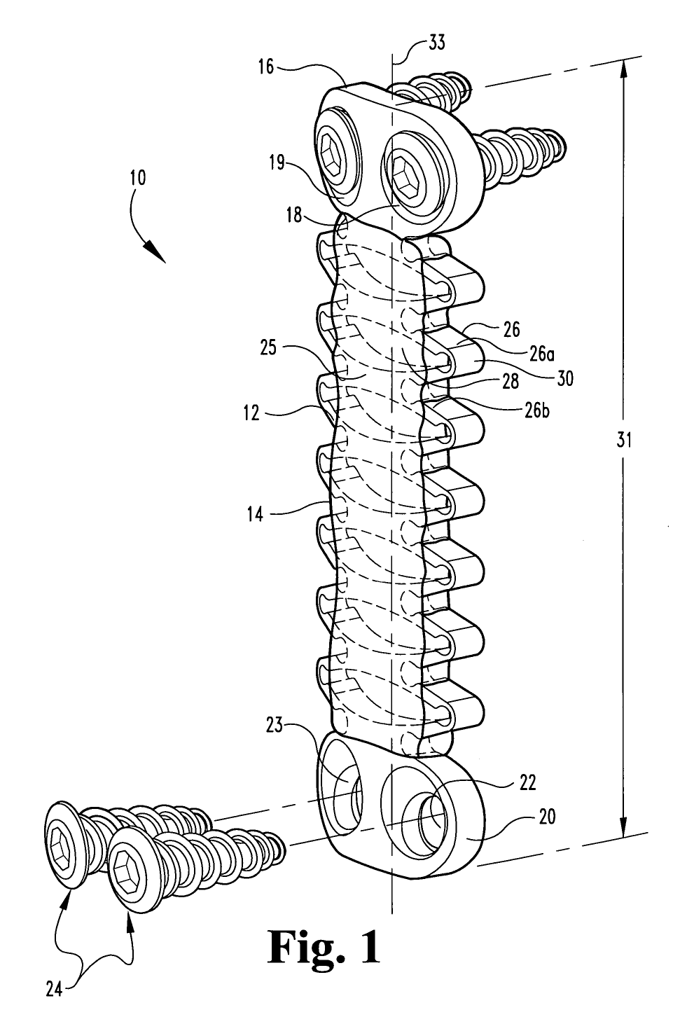

FIG. 1 is a perspective view of one embodiment of a bone fixation device 10 in accordance with the present invention. Bone fixation device 10 includes an elongate member 12 and a reinforcing component 14. Elongate member 12 can define a longitudinal axis 33 and can include a first end 16 that can be configured for attachment to one or more bony structures. In the illustrated embodiment, first end 16 includes first and second openings 18 and 19, respectively, through which a bone fastener can be inserted. Second end 20, opposite first end 16, can be similarly configured to be secured to bony structures and can include third and fourth openings 22 and 23. In alternative embodiments, either or both of first end and second end 20 can be configured with a single opening, a plurality of openings, or no openings. In any of the embodiments, elongate member 12 can be secured to bony structures using a variety of fasteners. Examples of suitable fasteners for use in the present invention include bone nails, staples, bone adhesives, bone screws, bone hooks, and the like. In the illustrated embodiment, elongate member 12 can be secured to one or more bony structures using one or more bone screws 24.

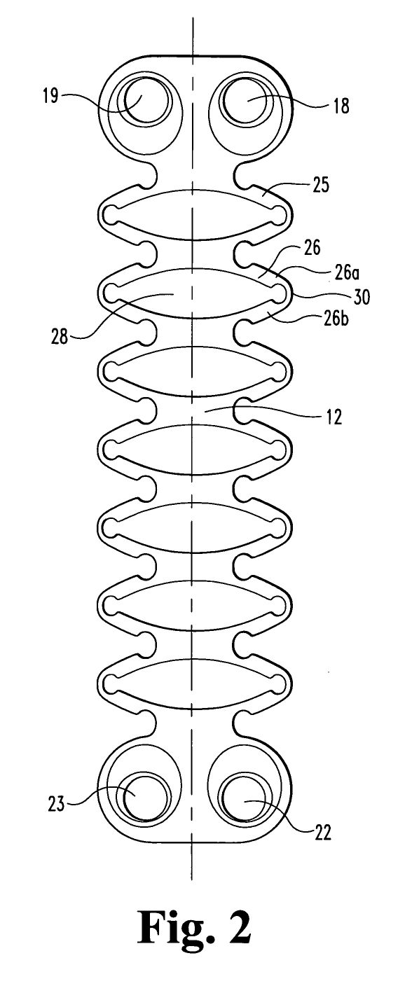

Referring additionally to FIG. 2, a first portion or bridge portion 25 introduces deformation and/or flexibility into the device 10. This flexibility can be exhibited by allowing movement in the longitudinal direction, i.e., translational movement. In other embodiments, this flexibility can arise or be derived from a rotational-torsional movement. A related bone plate is disclosed in U.S. Pat. No. 6,293,949, which is incorporated by reference in its entirety.

Bridge portion 25 is disposed between first end 16 and second end 20. Bridge portion 25 can be formed in whole or in part of a metal, polymer, or composite material that is flexible. In the illustrated embodiment, bridge portion 25 includes a plurality of structural members or an open network. In one embodiment, the open network can be provided to include a plurality of trusses 26 spaced from each other by voids 28. Each individual truss 26a and its neighbor 26b can be connected by a flexible junction 30. The length of bridge portion 25 and, consequently, the overall length of device 10 represented by reference 31 can vary depending upon whether the implant is subjected to expansive (tension) or compressive forces. This, in turn, allows the attached bone portions to move either closer together or further apart. In addition, or in the alternative, bridge portion 25 can twist about its longitudinal axis allowing the attached bone portions to rotate or twist relative to each other. It will be understood that in other embodiments, the network of voids is not restricted to bridge portion 25 but can be distributed along the entire length of elongate member 12.

The flexibility can be accomplished either by specific design configurations of the trusses 26 interspersed with voids 28 and connected with a variety of flexible junctions 30. Alternatively, the flexibility can be accomplished by the choice of material used to form the bridge portion. In preferred embodiments, the material selected to provide the structural features of the bridge portion includes resilient materials such as, without limitation, nitonal, titanium, titanium-vanadium-aluminum alloy, cobalt-chromium alloy, cobalt-chromium-molybdenum alloy, cobalt-nickel-chromium-molybdenum alloy, biocompatible stainless steel, tantalum, niobium, hafnium, tungsten, and alloys thereof; reinforced polymeric materials, poly(ether, ether, ketone) carbon (PEEK), poly (aryl, ether, ketone) (PAEK), and the like. Consequently, if desired, bridge portion 25 exhibits an elastic property and preferably performs analogous to a series of leaf springs stacked on top of each other.

It should be understood that other configurations can be used which impart the ability of the elongate member to be flexible both in compression and elongation as well as rotational directions. In one embodiment, truss 26 is provided to maintain rigidity and support for elongate member 12. Flexible junctures 30 can be formed of a similar material, albeit in much thinner dimensions, to allow neighboring truss portions 26a and 26b to approach one another and thus either elongate or decrease the distance between first end 16 and second end 20. Additionally, or in the alternative, flexible juncture 30 can allow a rotational movement such that truss 26a pivots about central elongate axis represented by reference line 33 while an adjacent truss portion 26b either remains stationary or translates rotationally to a lesser extent.

The reinforcing component 14 can be deposited on device 10. In the illustrated embodiment, reinforcing component 14 is deposited onto and into bridge portion 25. Consequently, reinforcing component 14 fills voids 28 interspersed between trusses 26a and 26b. The reinforcing component 14 serves to stiffen the bridge portion, and consequently, inhibit the translation and/or rotational movement afforded the device. This in turn can inhibit translation and/or rotational movement of the attached bone portions.

The reinforcing component can be formed or composed of a variety of rigid materials including, without limitation, resorbable polymeric materials, resorbable composite materials, and resorbable ceramic materials.

In one embodiment, reinforcing component 14 can include polymeric materials formed from oligomers, homopolymers, copolymers, and polymer blends that include polymerized monomers derived from l, d, or d/l lactide (lactic acid); glycolide (glycolic acid); ethers; acids; anhydrides; olefins, such as ethylene, propylene, butene-1, pentene-1, hexene-1,4-methylpentene-1, styrene, norbornene and the like; butadiene; polyfunctional monomers such as acrylate, methacrylate, methyl methacrylate; esters, for example, caprolactone and hydroxy esters; and mixtures of these monomeric repeating units.

Use of the term “copolymers” is intended to include within the scope of the invention polymers formed of two or more unique monomeric repeating units. Such copolymers can include random copolymers; graft copolymers; block copolymers; radial block, diblock, and triblock copolymers; alternating copolymers; and periodic copolymers. Use of the term “polymer blend” is intended to include polymer alloys, semi-interpenetrating polymer networks (SIPN), and interpenetrating polymer networks (IPN).

In a preferred embodiment, the reinforcing component 14 comprises a biodegradable polymeric material including: poly(amino acids), polyanhydrides, polycaprolactones, poly(lactic-glycolic acid), polyhydroxybutyrates, polyorthoesters, and polylactic acid, polyglycolic acid, and mixtures thereof. Specific examples of biodegradable materials for the present invention include poly (d,l-lactide) (PLDLA).

In other embodiments, the reinforcing component can comprise biodegradable ceramic materials and ceramic cements. Examples of biodegradable ceramic materials include: hydroxy apatite, hydroxyapatite carbonate, corraline, calcium phosphate, and tricalcium phosphate. Examples of biodegradable ceramic cements include calcium phosphate cement. Such calcium phosphate cements are preferably synthetic calcium phosphate materials that include a poorly or low crystalline calcium phosphate, such as a low or poorly crystalline apatite, including hydroxyapatite, available from Etex Corporation and as described, for example, in U.S. Pat. Nos. 5,783,217; 5,676,976; 5,683,461; and 5,650,176, and PCT International Publication Nos. WO 98/16268, WO 96/39202 and WO 98/16209, all issued to Lee et al. Use of the term “poorly or low crystalline” is meant to include a material that is amorphous, having little or no long range order and/or a material that is nanocrystalline, exhibiting crystalline domains on the order of nanometers or Angstroms.

In other embodiments, the reinforcing component can be formed of composite materials. Examples of composite materials include as a base material or matrix, without limitation: ceramics, resorbable cements, and/or biodegradable polymers listed above. Each of the base materials can be impregnated or interspersed with fibers, platelets, and particulate reinforcing materials including hydroxy apatite particles (HA)

In one form, the reinforcing component can comprise a resorbable, moldable material that can be molded at an elevated temperature and then allowed to set up into a hardened material at around body temperature, such as the material sold under the trade name BIOGLASS® discussed in WO 98/40133, which is incorporated by reference herein.

The reinforcing component of the present invention can be tailored to degrade at a predetermined or preselected rate. In preferred embodiments, the reinforcing component degrades at a rate comparable to the new bone ingrowth into the bone defect or bone fusion site. In particularly preferred embodiments, the reinforcing component has an in vivo half life of greater than three months, more preferably the in vivo half life of the reinforcing component is greater than six months; still more preferably the in vivo half life is greater than one year. By use of the term “half life”, it is understood that the degradation rate of the reinforcing component is such that the reinforcing component loses half of its initial mass in vivo, presumably due to resorption, degradation, and/or elimination.

The reinforcing component of the present invention provides a stabilizing component for the inventive device. This stabilizing component can provide rigidity and support for both the implanted orthopedic fusion device and, consequently, the attached bone structures. In use, the load supported by the bone fixation device and supported by the reinforcing component can vary. This allows the fixation device to become dynamizable, or change its support characteristics in vivo. This change in support characteristics can be particularly important for developing strong, new bone tissue at the bone defection or fusion site. This can prevent stress shielding of the new bone ingrowth and can minimize the risk for the development of pseudoarthrodesis.

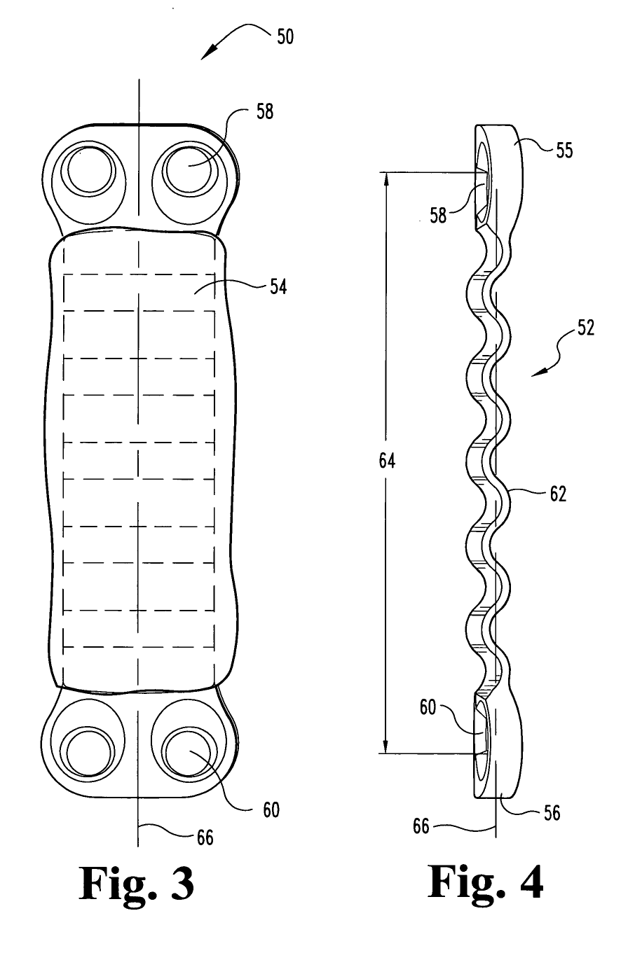

FIG. 3 is a plan view of yet another embodiment of a bone fixation device 50 in accordance with the present invention. Bone fixation device 50, similar to device 10, includes two basic components, an elongate member 52 and a reinforcing component 54.

FIG. 4 is an elongated side view of elongate member 52. Elongate member 52 includes a first end 55 and an opposite, second end 56 and a bridge portion 62 therebetween. Both first end 55 and second end 56 are provided with at least one opening 58 and 60, respectively, through which a bone fastener (not shown) can be inserted. In a preferred embodiment, bridge portion 62 is flexible, allowing movement of first end 55 relative to second end 56. This movement can be translational movement, i.e., increasing/decreasing the distance indicated by reference line 64 between first end 55 and second end 56, depending upon whether device 50 is subjected to tension or compressive force. In other embodiments, bridge portion 62 can allow for rotation or torsional movement of first end 55 relative to second end 56. This torsional movement can occur by a twisting rotation about the central axis 66 extending along the longitudinal direction of elongate member 52. In other embodiments, bridge portion 62 can allow first end 55 to bend closer to second end 56. In this embodiment, bridge portion 62 bends in a direction substantially orthogonal to longitudinal axis 66.

In the fixation device 50, prior to implantation, a reinforcing component 54 is engaged to at least a portion of bridge portion 62. In a preferred embodiment, the reinforcing component 54 envelopes or completely surrounds bridge portion 62. Consequently, bridge portion 62 is embedded within the reinforcing component. Reinforcing component 54 can be provided as has been discussed above for reinforcing component 14.

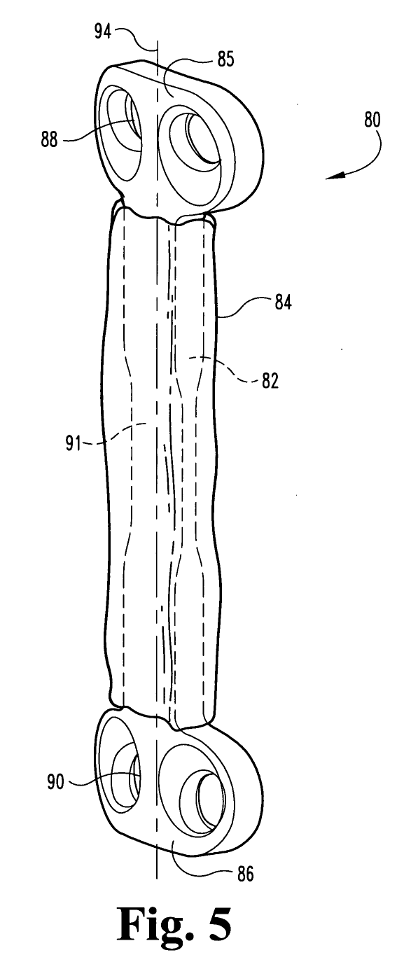

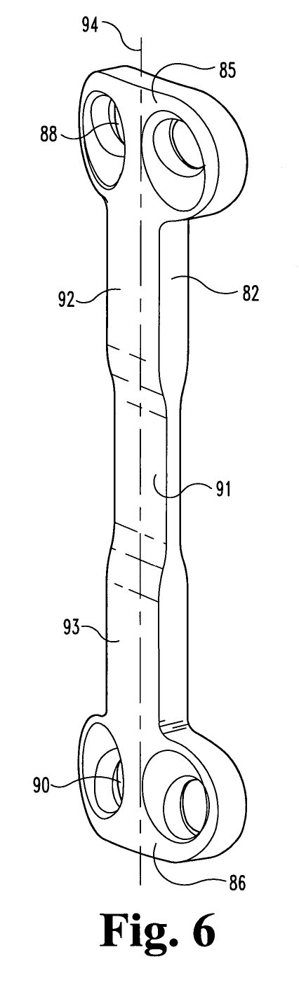

FIG. 5 illustrates still yet another embodiment of a bone fixation device 80 in accordance with the present invention. Bone fixation device 80 includes an elongate member 82 and a reinforcing component 84. Referring additionally to FIG. 6, elongate member 82 is illustrated absent reinforcing component 84. Elongate member 82 includes a first end 85 and an opposite, second end 86. Each of first end 85 and second end 86 include at least one and preferably a plurality of openings 88 and 90, respectively, through which a bone fastener can be inserted (not shown). Elongate member 82 includes a flexible or narrowed portion 91. In the illustrated embodiment, portion 91 is illustrated to have a substantially reduced cross-sectional area measured transverse to longitudinal axis 94 than that illustrated in adjacent portions 92 and 93 of the elongate member 82. Narrowed portion 91 impacts flexibility into fixation device 80. Consequently, narrowed portion 91 allows the elongate member 82 to bend substantially orthogonal to its longitudinal axis 94. Additionally or in the alternative, narrowed portion 91 allows the elongate member 82 to rotate or “twist” about the longitudinal axis 94 such that first end 85 is non planar with second end 86, i.e., first end 85 does not lie in the same plane as second end 86.

Elongate member 82 is at least partially encased within a reinforcing component 84. This reinforcing component reduces the flexibility of narrowed portion 91. This inhibits movement of first end 85 relative to second end 86. Reinforcing component 84 can comprise a material as has been described above for reinforcing components 14 and 54.

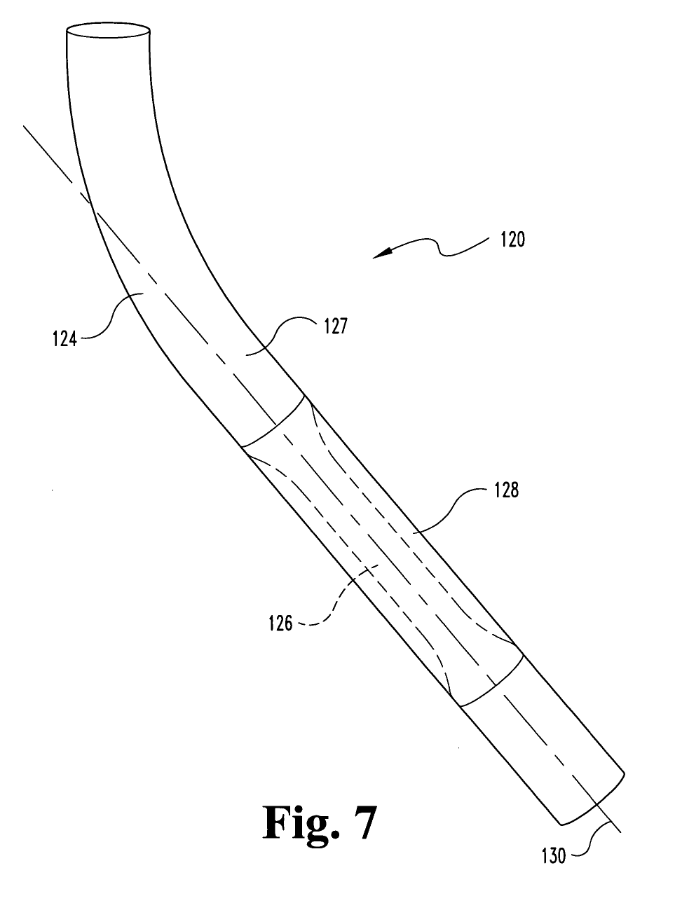

FIG. 7 illustrates still another embodiment of a bone fixation device 120 in accordance with the present invention. Bone fixation device 120 comprises an elongate member 124 illustrated as an elongated tubular member. Elongate member 124 can be provided, for example, as an implantable orthopedic rod such as, for example, a spinal rod or a cross-linking member between adjacent spinal rods. Elongate member 124 includes a bridge portion 126 represented in the illustration with dashed lines and disposed internal of a reinforcing section or component 128. Bridge portion 126 is illustrated as a rod portion having a smaller cross-sectional area (radius or diameter) than the adjacent, non-covered portion 127. In addition or in the alternative, bridge portion 126 can be provided with a plurality of holes or voids selectively sized and spaced to introduce flexibility into elongate member 124. Bridge portion 126 impacts a section or portion of elongate member 124 that can be more readily or easily bent proximate to this narrowed or bridge portion 126. Reinforcing component 128 encases at least a portion of bridge portion 126. Reinforcing section 128 can comprise a material substantially as has been described for reinforcing components 14 and 54. In this embodiment, it should be understood that reinforcing component is illustrated as a cylindrical sleeve that substantially surrounds bridge portion 126. In alternative embodiments, reinforcing section 128 can be provided as a partial sleeve that partially surrounds bridge portion 126. This partial sleeve can be perforate or imperforate and can include a variety of slits and other openings as desired. Additionally, reinforcing section 128 can be welded, glued, or over molded onto the elongate member 124. In other embodiments, reinforcing section 128 can be provided to be readily separable from bridge portion 126 and/or elongate member 124. For example, reinforcing section 128 can be provided to translate along the longitudinal axis 130 of elongate member 124; i.e., slide up the elongate member 124 to reveal the underlying bridge portion 126.

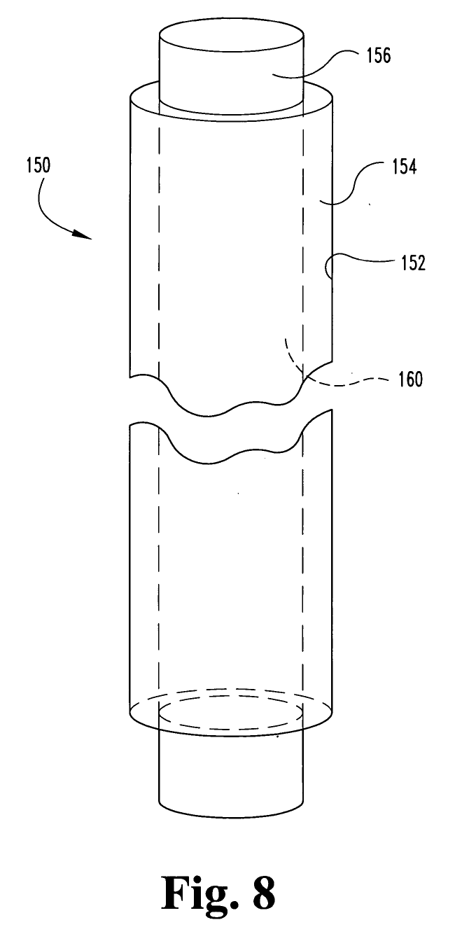

FIG. 8 is still yet another embodiment of a bone fixation device 150 prepared in accordance with the present invention. In the illustrated embodiment, bone fixation device 150 includes an outer cylindrical rod 152 provided as an elongate member 154. Elongate member 154 is provided with a hollow interior or lumen into which a reinforcing component 156 has been inserted.

In preferred embodiments, elongate member 154 is provided as a flexible conduit that can be bent and shaped as desired. The elongate member 154 can be pre-bent by the manufacturer or bent by the surgeon either immediately prior to or during surgery. Reinforcing component 156 is provided to be disposed in the interior section 160 of elongate member 154.

The reinforcing component 156 can comprise a material as has been described for reinforcing components 14, 84, and 128. Furthermore, reinforcing component 156 can be separable from elongate member 154. Elongate member 154 and reinforcing component 156 can be provided to the surgeon as separate components that can be combined by sliding elongate member 154 over reinforcing component 156 either prior to or during surgery. Alternatively, bone fixation device 150 can be provided to the surgeon as a one-piece unit that is ready for implantation or that can be molded, bent, or deformed as desired and/or as deemed medically expedient by the orthopedic surgeon. Furthermore, reinforcing component 156 inhibits the flexibility of elongate member 154. Consequently, when combined together, reinforcing component 158 and elongate member 154 provide a stiff rod that inhibits both movement, either translational, rotational, or torsional.

In additional embodiments, elongate member 154 can be secured to one or more bone portions to induce bone fusion or arthrodesis. This can be accomplished using a variety of techniques including gluing, staples, bone screws, hooks, and the like as known in the art. Bone fixation devices, elongate members, and reinforcing components described in the present invention can be fabricated by a wide variety of techniques, including injection molding, extrusion molding, over molding, blow molding, transfer molding, and the like.

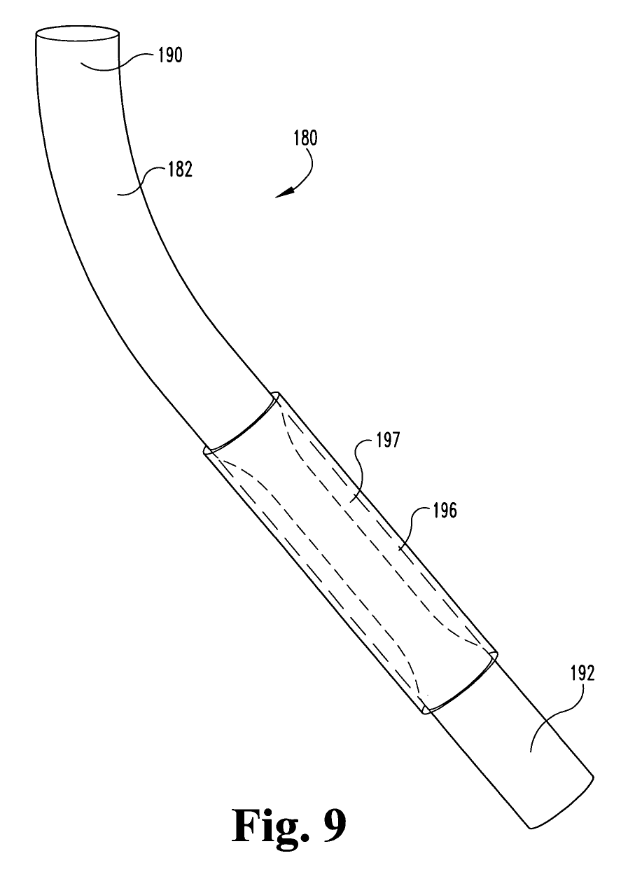

FIG. 9 is a perspective view of another embodiment of a bone fixation device 180 for use in accordance with the present invention. Bone fixation device 180 includes an elongate member 182 and a reinforcing component 196. Elongate member 182 can be attached to two or more bone portions. A first end 190 of member 182 can be attached to a first bone portion, such as a first vertebra, using a bone fixation device such as a bone screw. Similarly, second end 192 of member 182 can be secured to a second vertebral body using a bone fastener.

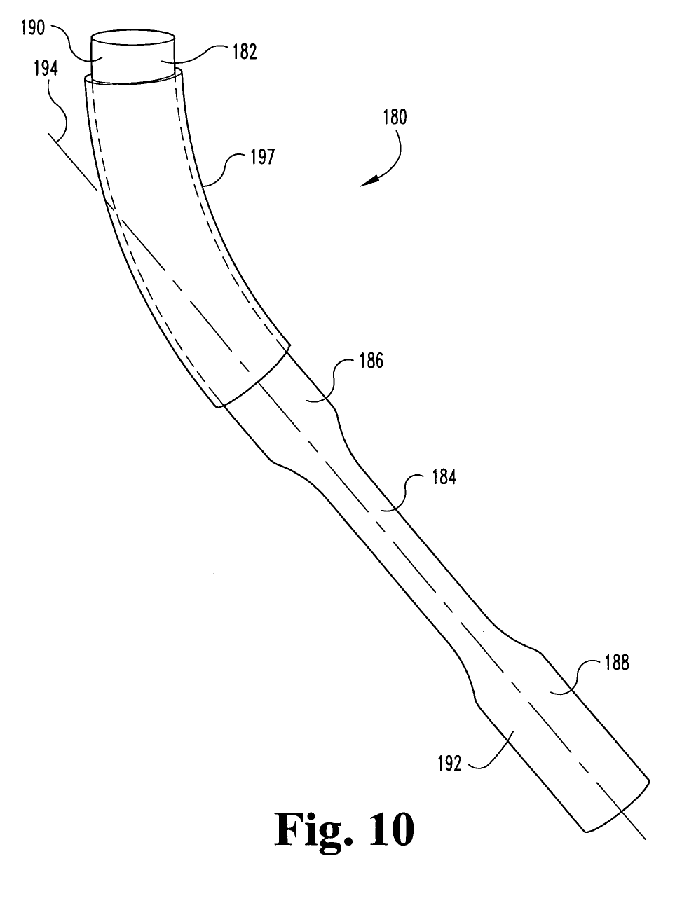

Bone fixation device 180, similar to device 120, includes an elongate member 182, which is illustrated as an elongate rod. (See also FIG. 10.) Elongate member 182 includes a narrowed portion 184 that has a diameter that is substantially reduced from the remaining portions of elongate member 182. For example, narrowed portion 184 has a diameter that is substantially smaller than that found in neighboring portions 186 and 188 of elongate member 182. The narrowed portion 184 allows the elongate member 182 to become flexible, i.e., it can be bent and/or twisted to allow translational and/or rotational-torsional movement. For example, narrowed portion 184 can allow a first end 190 of member 182 to bend toward second end 192 substantially orthogonal to the longitudinal axis 194. Additionally, narrowed portion 184 can allow first end 190 and/or second end 192 to twist about axis 194 to allow rotational-torsional rotation.

In preferred embodiments, elongate member 182 is provided as a spinal rod, a connecting member between adjacent spinal rods, and/or a spinal rod and a bone fastener and/or an orthopedic implant to promote spinal fusion. Reinforcing component 196 is provided as a movable sleeve 197 about elongate member 182. Movable sleeve 197 can be provided in a first position illustrated in FIG. 9 wherein sleeve 197 is disposed adjacent to or around narrowed portion 184. In this configuration, sleeve 197 inhibits deformation of narrowed portion 184 and, consequently, elongate member 182 by preventing either bending, i.e., movement substantially orthogonal to longitudinal axis 194 and/or rotational-torsional movement about axis 194. As seen in FIG. 10, sleeve 197 is slidably disposed about elongate member 182. Consequently, sleeve 197 is provided to have a diameter that is larger than the external diameter of elongate member 182. Alternatively, elongate member 182 can be provided with at least a portion that has an external diameter smaller than the internal diameter of sleeve 197. When thus configured, sleeve 197 can be slidably disposed about elongate member 182. As shown more fully in FIG. 10, sleeve 197 can slide either upward or downward on elongate member 182 and expose the narrowed portion 184. When thus exposed, narrowed portion 184 can be deformed to allow the attached bone portions to have either translational and/or rotational-torsional movement in respect to one another.

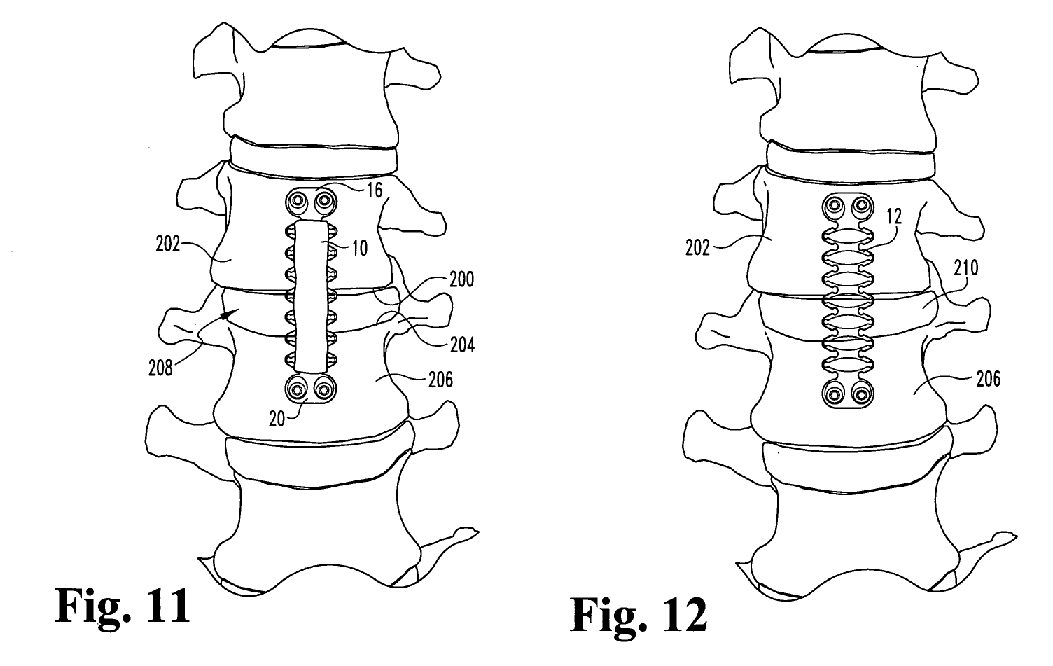

In use, any of the bone fixation devices 10, 50, 80, 120, 150, and 180 can be used to secure and treat bone defects. For example, as illustrated in FIG. 11, the bone fixation device 10 can be used to treat a spinal defect. In this specific illustration, the spinal defect occurs either on the inferior end plate 200 of vertebra 202 and/or the superior end plate 204 of vertebra 206. The surgeon can perform either a full or partial discectomy if desired and if the defect occurs in the nucleus pulposa and/or spinal disc structure. The discectomy can include either replacing the disc with a disc prosthesis and/or inserting a spinal spacer between the affected vertebrae, which spinal spacer can induce bone fusion or not, as desired. The illustration uses bone fixation device 10 by attaching its first end 16 to first vertebra 202 and attaching its second end 20 to an adjacent, second vertebra 206. Device 10 maintains the desired disc space height 208 and maintains vertebrae 202 and 206 in a rigid confirmation relative to one another.

Referring additionally to FIG. 12, it can be observed that over time or under selected conditions, the reinforcing component 14 of device 10 has been eroded or degraded away, leaving the elongate member 12. In this embodiment, it can be observed that a prosthetic disc 210 has been inserted between vertebra 202 and 206. Consequently, it is desirable to maintain the relative movement of 202 in relation to vertebra 206. The flexibility of elongate member 12 allows limited mobility of the two vertebrae either by translational and/or rotational-torsional movement relative to each other.

In addition or in the alternative, it may be desirable to promote bone fusion between the adjacent vertebrae or between any bone portions on either side of a bone defect. In this embodiment, it may be desirable to include a bone growth material such as an osteoinductive or an osteoconductive material. For example, it may be desirable to introduce a osteogenic factor such as a bone morphogenic protein (BMP). Examples of bone growth materials include an osteoinductive factor, such as an osteoinductive protein or a nucleotide or a nucleotide sequence encoding an osteoinductive protein operably associated with a promoter (e.g., provided in a vector such as a viral vector), for example a bone morphogenetic protein or a gene encoding the same operationally associated with a promoter which drives expression of the gene in the animal recipient to produce an effective amount of the protein. The bone morphogenic protein (BMP) in accordance with this invention is any BMP able to stimulate differentiation and function of osteoblasts and osteoclasts. Examples of such BMPs are BMP-2, BMP-4, and BMP-7, more preferably rhBMP-2 or rhBMP-7, most preferably, rhBMP-2. Purified recombinant BMPs are preferred for use in the inventive compositions for their provision of high osteoinductive potentials. BMP gene sequences and methods for producing recombinant and naturally-derived BMPs are known in the art, and for additional information on this subject reference may be made, for instance, to U.S. Pat. Nos. 5,108,753; 5,187,076; 5,366,875; 4,877,864; 5,108,922; 5,116,738; 5,013,649; 5,106,748; and 4,294,753; and International Publication Nos. WO93/00432; WO94/26893; and WO94/26892. The osteoinductive factor may also be LIM mineralization protein (LMP) or a suitable vector incorporating a gene encoding the same operably associated with a promotor, as described in WO99/06563 (see also genbank accession No. AF095585). When such vectors are employed as osteogenic factors in accordance with the invention, they are preferably delivered in conjunction with cells, for example autologous cells from the recipient of the implant. Most preferably the vector is delivered in conjunction with autologous white blood cells derived from bone marrow or peripheral blood of the recipient.

The osteogenic factor will be incorporated in an amount which is effective to stimulate the formation of bone within the animal recipient. In more preferred compositions incorporating protein osteogenic factors, the osteogenic factor will be incorporated in a weight ratio of about 1:100 to about 1:1000 relative to the overall composition, more preferably about 1:100 to about 1:500. As will be understood, when the osteogenic factor comprises a nucleotide sequence, sufficient amounts of the delivery vehicle (vector) will be incorporated to cause significant transduction of cells, so as to cause the generation of sufficient protein at the site to induce bone formation. The orthopedic devices of the present invention can be used by themselves or in conjunction with one or more known orthopedic devices as deemed medically prudent. Additionally or in the alternative, the present invention can be used with one or more devices disclosed in co-pending U.S. patent application Ser. No. ______ filed on Oct. 21, 2003 and entitled “Apparatus and Method for Providing Dynamizable Translation to a Spinal Construct”, Attorney Docket No. 4002-3273, which is hereby incorporated by reference.

The bone growth material may be used singly or in combination with one or more spacers, bone plates, screws, fasteners, and the like. In this alternative, the reinforcing component of the bone fixation device can be prepared to erode or biodegrade at a selected or predetermined rate. The rate of degradation can be selected to allow new bone growth to occur under conditions optimal to generate a dense cortical bone bridge between the bone portions.

While the invention has been illustrated and described in detail in the drawings and foregoing description, the same is considered to be illustrative and not restrictive in character, it is understood that only the preferred embodiments have been shown and described and that all changes and modifications that come within the spirit of the invention are desired to be protected. Any reference to a specific directions, for example, references to up, upper, down, lower, and the like, is to be understood for illustrative purposes only or to better identify or distinguish various components from one another. These references are not to be construed as limiting in any manner to the orthopedic device and/or methods for using the orthopedic device as described herein.

All publications, patents, and patent applications cited in this specification are herein incorporated by reference as if each individual publication, patent, or patent application was specifically and individually indicated to be incorporated by reference and set forth in its entirety herein.

Unless specifically identified to the contrary, all terms used herein are used to include their normal and customary terminology. Further, while various embodiments of medical devices having specific components and structures are described and illustrated herein, it is to be understood that any selected embodiment can include one or more of the specific components and/or structures described for another embodiment where possible.

Further, any theory of operation, proof, or finding stated herein is meant to further enhance understanding of the present invention and is not intended to make the scope of the present invention dependent upon such theory, proof, or finding.

1. An orthopedic device for securing two or more bone portions, said device comprising:
an elongate member configured for engagement to the two or more bone portions and allowing translational or rotational movement for a first one of the two or more bone portions relative to a second one of the two or more bone portions;
a reinforcing component composed of a biodegradable material and engaged to the elongate member to inhibit the translational or rotational movement for a first one of the two or more bone portions relative to a second one of the two or more bone portions; and
at least one bone fastener for fixedly securing the elongate member to at least one of the two or more bone portions.

2. The device of claim 1, wherein at least some of the load on said device is transferred to two or more bone portions as said reinforcing component degrades.

3. The device of claim 1, wherein said elongate member allows restricted translational or rotational movement of two or more bone portions after said reinforcing component degrades.

4. The device of claim 1, wherein the elongate member is composed of a biocompatible metal.

5. The device of claim 1, wherein the elongate member is formed of an elastic material.

6. The device of claim 1, wherein said elongate member is composed of a biocompatible metal or a metal selected from the group consisting of: nitinol, titanium, titanium-vanadium-aluminum alloy, cobalt-chromium alloy, cobalt-chromium-molybdenum alloy, cobalt-nickel-chromium-molybdenum alloy, biocompatible stainless steel, tantalum, niobium, hafnium, tungsten, and alloys thereof.

7. The device of claim 1, wherein said reinforcing component degrades within two years while said elongate member remains engaged to the two or more bone portions.

8. The device of claim 1, wherein said reinforcing material has an initial mass upon implantation and the reinforcing material degrades to less than half its initial mass within one year.

9. The device of claim 8, wherein said elongate member allows restricted translational or rotational movement of two or more bone portions after said reinforcing component degrades.

10. The device of claim 1, wherein said reinforcing material retains at least half of its initial mass for a time period of greater than one year.

11. The device of claim 1, wherein said reinforcing element is composed of a material selected from a group consisting of: poly(amino acids), polyanhydrides, polycaprolactones, polylactates, polyglycolates, poly(lactic-glycolic acid), polyorthoesters, and blends thereof.

12. The device of claim 1, wherein the elongate member is a bone plate.

13. The device of claim 12 wherein the bone plate is configured with a plurality of voids.

14. The device of claim 13 wherein the reinforcing material is disposed in the plurality of voids.

15. The device of claim 12 wherein the bone plate is imperforate.

16. The device of claim 12 wherein the reinforcing material encases at least a portion of the bone plate.

17. The device of claim 12 wherein the bone plate comprises a first portion configured to allow the bone plate to be deformed.

18. The device of claim 17 wherein the bone plate comprises a second portion adjacent to the first portion, where said second portion is configured to resist deformation.

19. The device of claim 18 wherein the first portion has a first cross sectional area and the second portion has a second cross sectional area greater than the first cross sectional area.

20. The device of claim 19 wherein the first portion comprises a plurality of voids and the second portion is imperforate.

21. The device of claim 20 wherein the reinforcing component is disposed in the plurality of voids.

22. The device of claim 12, wherein said reinforcing material has an initial mass upon implantation and the material degrades to less than half its initial mass within one year.

23. The device of claim 12, wherein said reinforcing material retains at least half of its initial mass for a time period of greater than one year.

24. The device of claim 1, wherein the elongate member is an orthopedic rod.

25. The device of claim 24, wherein the orthopedic rod is a spinal rod.

26. The device of claim 24, wherein the spinal rod is configured with a plurality of voids.

27. The device of claim 24, wherein the reinforcing material is disposed in the plurality of voids.

28. The device of claim 24, wherein the orthopedic rod is imperforate.

29. The device of claim 24, wherein the reinforcing material encases at least a portion of the orthopedic rod.

30. The device of claim 24, wherein the orthopedic rod comprises a first portion configured to allow the orthopedic rod to be deformed.

31. The device of claim 30, wherein the orthopedic rod comprises a second portion adjacent to the first portion, where said second portion is configured to resist deformation.

32. The device of claim 31, wherein the first portion has a first cross sectional area and the second portion has a second cross sectional area greater than the first cross sectional area.

33. The device of claim 30, wherein the first portion comprises a plurality of voids and the second portion is imperforate.

34. The device of claim 33 wherein the reinforcing component is disposed in the plurality of voids.

35. The device of claim 24, wherein the orthopedic rod is hollow and defines an interior lumen and wherein the reinforcing material is disposed in the interior lumen.

36. The device of claim 24, wherein said reinforcing material has an initial mass upon implantation and the material degrades to less than half its initial mass within one year.

37. The device of claim 24, wherein said reinforcing material retains at least half of its initial mass for a time period of greater than one year.

38. The device of claim 1 wherein the elongate member comprises means for allowing movement of the first bone portion relative to the second bone portion.

39. A method for treating a bone defect, said method comprising fixedly attaching the device of claim 1 to two or more bone portions.

40. A method for treating a bone defect, said method comprising
providing an orthopedic device including an elongate member configured to be deformable in vivo, and a reinforcing component encasing at least a portion of the elongate member, said reinforcing component comprising a biodegradable material, formulated to inhibit deformation of the elongate member; and
securing a first end of the elongate member to a first bony structure and securing a second end of the elongate structure to a second bony structure.

41. The method of claim 40 wherein securing comprising fixedly securing the first end to the first bony structure using a bone screw, a suture or a bone cement.

42. The method of claim 40 wherein said securing comprises securing a first end of the elongate member to a first vertebra and securing the second end of the elongate member to a second vertebra.

43. The method of claim 40 comprising deforming the elongate member into a first configuration prior to securing the first end to a first bony structure.

44. The method of claim 43 comprising combining the reinforcing component and the elongate member after the elongate member has been deformed.

45. The method of claim 44 wherein the reinforcing component comprises a material selected from the group consisting of: poly(amino acids), polyanhydrides, polycaprolactones, polylactates, polyglycolates, poly(lactic-glycolic acid), polyorthoesters, and blends thereof.

46. The method of claim 40 comprising combining an bone growth material with the orthopedic device to promote arthrodesis.
